(19) United States
(12) Patent Application Publication
(10) Pub. No.: US 2003/0102246 A1
(43) Pub. Date: Jun. 5, 2003

(54) Method for improving the stability of stored and/or used light-sensitive therapeutic systems or components thereof

(75) Inventor: Achim Berthold, Andernach (DE)

Correspondence Address: JORDAN AND HAMBURG LLP, 122 EAST 42ND STREET, SUITE 4000, NEW YORK, NY, 10168, US

(73) Assignee: LTS Lohmann Therapie-Systeme AG, Andernach (DE)

(21) Appl. No.: 10/308,442

(22) Filed: Dec. 3, 2002

Related U.S. Application Data
(63) Continuation-in-part of application No. 09/937,121, filed on Sep. 20, 2001, now abandoned.
Application No. 09/937,121 is a 371 of international application No. PCT/EP00/01986, filed on Mar. 8, 2000.

(30) Foreign Application Priority Data
Mar. 20, 1999 (DE) 199 12 623.2

(51) Int. Cl. B65D 1/09; B65D 83/04; B65D 85/42; A61K 31/44

(52) U.S. Cl. 206/528; 514/355

(57) ABSTRACT

A process for increasing the stability during storage and/or application of light-sensitive therapeutic systems and/or components thereof, such as active ingredients or auxiliary substances, using light-stability agents absorbing or reflecting electromagnetic waves is characterized in that absorption or reflection agents are used whose absorption or reflection spectrum comprises that wavelength range which is responsible for the instability of the light-sensitive material or components thereof.

The invention relates to a process for increasing the stability during storage and/or application of light-sensitive therapeutic systems or components thereof, such as active substances or auxiliary substances, using light stability agents absorbing or reflecting electromagnetic waves. The invention further relates to administration forms such as therapeutic systems or the components thereof wherein the stability of light-sensitive parts or components is increased by means preventing the access of stability-impairing electromagnetic radiation or of other influences such as, for instance, aerial oxygen.

The causes of the instability of an administration form are of a twofold nature. On the one hand, it is the lability of the pharmaceutical active agents or the auxiliary agents themselves, which ultimately results from their chemical or physico-chemical structure, on the other hand, it is the external factors, such as temperature, humidity, atmospheric oxygen and light, which induce or accelerate reactions diminishing efficacy.

The degree to which these factors become effective is to a large extent dependent on the galenic composition of the preparations.

Generally, it is possible to distinguish between physical, chemical and microbial instability. Physical stability-impairing processes can be, for example:

a change in the crystal structure

a change in the state of distribution

a change in the consistency or state of aggregation

a change in the relations of solubility, or

a change in the relations of hydration

Stability-impairing chemical reactions are, for example:

hydrolysis

oxidation

reduction

steric rearrangement

decarboxylation or polymerisation.

Frequently, it is practically impossible to assign a particular instability exactly to one of the above categories since in many cases complex interactions are involved the results of which can be determined or perceived only through their final effect.

In known measures of stabilisation, the protection against light is of great significance. Action of light can impair the stability of an active substrate itself, but also the stability of the auxiliary agents employed. Thus, for instance, storage of oxidation-sensitive substances in light-proof or partially translucent vessels, e.g. porcelain jars or vessels of brown glass, is well known and absolutely imperative in order to ensure sufficient storage stability. Investigations have shown that it is always only a particular spectral range of light which is responsible for light-induced instabilities. It has further been shown that the most effective protection against light is attained by substances or measures the absorption maxima of which lie in the region of those wavelengths which are mainly responsible for the degradation. This fact will in the following be illustrated in detail by way of examples:

In the case of the example of the very light-sensitive vitamine A acid—an active agent used for treating acne vulgaris—it was possible to show that the instability thereof is caused mainly by electromagnetic waves having a wavelength of 400 nm. It was further shown that by using a yellow colourant having its absorption maximum in the range of the wavelength mentioned, the rate of degradation of the active substance can be substantially reduced. Other measures, for instance, the use of light-stability or screening agents, which absorb UV-A or UV-B rays and are commonly used in sun cremes, did not yield the desired results. (Briseart, M.; Plaizier-Vercammen; J. A.; Investigation on the Photostability of Tretinoin Lotion and Stabilization with Additives; Proc. 2nd World Meeting on Pharmaceutics, Biopharmaceutics and Pharmaceutical Technology; AGPI/APV, Paris, May 25-28, 1998, 1231-1232).

1,4-Dihydropyridine derivatives are known to be very light-sensitive. 1,4-Dihydropyridines are medicinally used as so-called calcium channel blockers. The active substance group serves to treat hypertension and the coronary diseases. Examples are nifidipine (Adalat®), nitrendipine (Bayo-tensin®), nimodipine (Nimotop®), felodipine (Modip®), nicardipine (Antagonil®), lacidipine (Motens®), nisoldipine (Baymycardo), nilvadipine (Escor®), isradipine (Lomir®), amlodipine (Norvasc®). Due to their physico-chemical properties, 1,4-dihydropyridine derivatives are suitable for transdermal application.

It is further known that the type of packaging has a strong influence on the stability of the 1,4-dihydropyridine derivates. The stability can be increased by addition of light-absorbing or light-reflecting additives. In the case of the example of the yellow-coloured nifidipine it was possible to prove the influence differently coloured packages have on stability. The best results were achieved where active substance-containing tablets were packed in a green blister pack. The protection against light weakened increasingly from yellow to red to orange. No protection was obtained from blue or colourless press-through packs, so-called-blister packs. The use of UV-A radiation-absorbing substances did not lead to an improvement.

It results therefrom that the best protection is ensured by films/foils whose absorption spectrum comprises that wave-length which is responsible for the degradation of the active substance.

It was possible to further increase the protection afforded by such coloured films/foils by incorporating opalescent substances such as titanium dioxide.

A large number of pharmaceutical substances which are components of transdermal therapeutic systems or formulations show light-sensitive behaviour and are degraded when exposed for a prolonged period to the influence of light. To increase their stability, in particular during storage, it is therefore necessary to provide special light protection. To this end, different measures have been known, and described in the literature.

WO 91/09731 describes a packaging material suitable for long-term storage of nicotine preparations. To produce the packaging material, a laminate is used which serves as a barrier. In this function, the laminate is intended to neutralise the influence of different external factors, such as air, water and/or light, which impair the stability of nicotine.

U.S. Pat. No. 5,008,110 describes a transdermal patch used, for example, for administration of buprenorphine. A characteristic feature is that this transdermal therapeutic system (TTS) is encapsulated in a hermetically sealed compartment protecting the formulation from environmental factors.

The protective action is obtained by using materials that are impermeable to air, water and light. This measure increases the stability of the preparation and ensures efficacy.

U.S. Pat. No. 4,597,961 describes a TTS for administering nicotine. This TTS consists of a carrier film, a film permeable to nicotine, a matrix containing the nicotine, and an adhesive for attaching the TTS on the skin. A characteristic feature is that the carrier film is impermeable to air, water and light. The impermeability to air and light protects the nicotine against degradation, and the water-impermeability prevents nicotine diffusion.

The measures described above offer a general protection. Films/foils or laminates are used which constitute a part of the primary packaging or of the therapeutic systems. The characteristic feature of these measures is that they do not offer specific protection but rather aim at protecting the therapeutic systems against environmental influences in general. In this context, the factors air, water and light have been mentioned.

It is the object of the present to provide a process for increasing the stability during storage and/or application of light-sensitive therapeutic preparations, systems or of the components thereof such as active or auxiliary substances, said process using light-stability agents absorbing or reflecting electromagnetic waves, in order to ensure—especially in therapeutic systems for application of active substance to or through the skin—the stability of light-sensitive components by providing a respective specific protection against degradation caused by detrimental factors, such as aerial oxygen, water and/or light.

To achieve this object, it is proposed with the present invention that for protection of the therapeutic preparations, systems or their components such as active or auxiliary agents there are employed absorbing or reflecting agents whose absorption or reflection spectrum, respectively, comprises that wavelength range which is responsible for the instability of the light-sensitive substance or the components thereof, respectively.

The inventive measure described affords optimal protection since the noxae responsible for instability are kept away specifically.

The invention will in the following be illustrated by way of an example:

### EXAMPLE

### Increasing the Stability by Use of Coloured Polymers

As a representative of the very light-sensitive 1,4-dihydropyridine derivatives, lacidipine is used. By way of the example of lacidipine it is possible to show the influence the absorption spectrum of a polymer, for instance, one based on polypropylene, has on the stability of the 1,4-dihydropyridine derivative.

To carry out the experiment, the lacidipine was dissolved in a solvent. Since dissolved lacidipine is very sensitive to electromagnetic-radiation, it was possible to clearly determine the influence of the polymers examined. The lacidipine solution was filled in differently coloured vessels of polypropylene and exposed to daylight for a defined period of time. The vessels in this context served as models for a coloured film. After the vessels had been exposed to a certain radiation (for a time of 6 to 8 hours), the lacidipine content of the samples was determined. On the basis of the known initial concentration and the detected active substance amounts it was possible to make a statement on the protective action of the polymers used. The results showed that the most efficient light protection is ensured if the absorption spectrum of the polymer employed comprises that region of wavelengths which is responsible for the instability of the lacidipine. The results are listed in the following Table 1:

TABLE 1

| | | Amount of active |
|---|---|---|
| | | substance after |
| | | irradiation |
| Colour of the | | [% of initial |
| polymer | Absorption range | amount] |
| green, clear | <325 nm; >800 nm | 12.40 |
| transparent, clear | <300 nm | 14.79 |
| orange, klar | 550 nm-750 nm | 15.65 |
| transparent, cloudy | <300 nm | 21.05 |
| blue, clear | <325 nm; | 23.46 |
| | 500 nm-700 nm | |
| yellow, clear | <350 nm; | 29.32 |
| | >700 nm | |
| brown, clear | <500 nm | 98.93 |
| aluminized | lichtundurchlässig | 99.36 |

The absorption spectrum of lacidipine contains three maxima [238.4 nm; 282.8 nm; 367.4 nm]. These wavelength regions determine the light-sensitivity of the active substance. As can be seen from the table, only the brown-coloured (respectively, the aluminized) polypropylene includes the entire absorption spectrum of the lacidipine and thus affords sufficient protection from light. It can thus be concluded that to ensure maximum stability in the present case brown or aluminized polymers should be used.

Further embodiments of the invention are provided in accordance with the sub-claims.

Finally, the invention comprises an administration form in which the stability of light-sensitive parts or components is substantially increased by means for preventing the access of stability-impairing components such as air, water and/or light, by the fact that said means include materials, such as glas, films/foils, polymers etc. whose absorption or reflection spectrum comprises that region of wavelengths which is responsible for the degradation of active substances or auxiliary substances, and which are impermeable at least to the access of air and light.

1. A process for increasing the stability during storage and/or application of a product which is a light-sensitive therapeutic system and/or a component thereof, the product including a light-sensitive material, comprising protecting the light-sensitive material by means for absorbing or reflecting electromagnetic waves of all wavelengths responsible for degradation of the light-sensitive material, wherein the means of absorbing or reflecting comprises a colored polymer or colored film or colored foil or laminate composed thereof or colored glass.

2. The process according to claim 1, wherein the colored polymer or colored film or colored foil or laminate comprised thereof or colored glass contains another lights absorbing or light-reflecting material.

3. The process according to claim 1, wherein the colored polymer or the colored film or the colored foil or the laminate comprised thereof or the colored glass comprises packaging for the therapeutic system or for a component thereof.

4. The process according to any on of claims 1 to 3, wherein the colored polymer or the colored film or the colored foil or the laminate comprised thereof or the colored glass have absorption or reflection maxima within the region of those wavelengths responsible for the degradation of the light-sensitive material.

5. The process according to claim 1, wherein the colored polymer or the colored film or the colored foil or the laminate comprised thereof or the colored glass is translucent.

6. The process according to claim 1, wherein the colored polymer or the colored film, or the colored foil or the laminate comprised thereof or the colored glass is impermeable to humidity and ambient oxygen.

7. The process according to claim 1, wherein the product comprises a membrane and an active substance, the membrane controlling the release rate of the active substance.

8. The process according to claim 1, wherein the colored polymer or the colored film or the colored foil or the laminate comprised thereof comprises a backing layer of a therapeutic system.

9. The process according to claim 1, wherein the therapeutic system or component thereof comprises an active substance or auxiliary substance containing the colored polymer.

10. The process according to claims 1, wherein at least one component selected from the group consisting of colored polymer, colored film, colored foil or the laminate comprised thereof comprises a pressure-sensitive adhesive layer and/or a removable protective layer of a therapeutic system.

11. The process according to claim 1, therein the light-sensitive material is a 1,4-dihydropyridine.

12. A combination of (a) a therapeutic preparation, system or a component thereof containing a light-sensitive material and (b) a colored polymer or a colored film or a colored foil or laminate comprised thereof or a colored glass which absorbs or reflects electromagnetic waves of all wavelengths responsible for degradation of the light-sensitive material and which is impermeable at least to ambient oxygen.

13. The combination of claim 12, wherein the colored polymer or the colored film or the colored foil or the laminate comprised thereof or the colored glass is translucent.

14. The combination of claim 12 or claims 13 , wherein the light sensitive-material is a 1,4-dihydropyridine.
